(19) United States
(12) Patent Application Publication
(10) Pub. No.: US 2010/0216114 A1
(43) Pub. Date: Aug. 26, 2010

(54) METHOD AND APPARATUS FOR DETERMINATION OF COMPONENTS IN ROOT CROPS

(75) Inventors: Frank Friedhoff, Einbeck (DE); Elke Hilscher, Einbeck (DE)

Correspondence Address: PATENT CENTRAL LLC;Stephan A. Pendorf, 1401 Hollywood Boulevard, Hollywood, FL, 33020, US

(73) Assignee: KWS Saat AG, Einbeck (DE)

(21) Appl. No.: 12/620,764

(22) Filed: Nov. 18, 2009

(30) Foreign Application Priority Data
Feb. 26, 2009 (DE) 10 2009 010 438.0

(51) Int. Cl. C12Q 1/00; C12M 1/34

(52) U.S. Cl. 435/4; 435/287.1

(57) ABSTRACT

The invention relates to a process for the determination of components in root crops as well as a device for performing the process. Root crops of a plot are prepared in such a way that becomes possible, using near-infrared spectroscopy, to determine the content of substances with a high analytical accuracy and short analysis time and wherein the recorded data concerning the identity and quantity of ingredients are representative of all root crops of a plot.

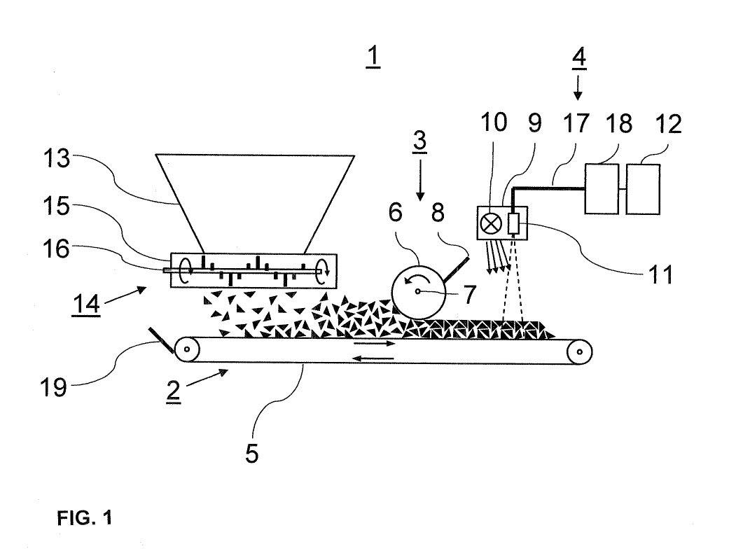

The invention relates to a process for the determination of components in root crops as well as a device for performing the process.

In the cultivation of root crops, measurement of contents plays an important role. Root crops, for the purposes of this patent application, are root crops such as sugar beets, fodder beet, and turnips, as well as tubers such as potatoes and Jerusalem artichokes. Cultivation involves a continuous, systematic selection of suitable root crops with respect to, for example, yield or disease tolerance. To be able to exercise such a selection, the contents of these crops are regularly analyzed. This is associated with a high investment in terms of labor and expense. Ultimately, however, the success of a breeding program is contingent upon the rapid and reliable analysis of the contents of root crops.

For culturing purposes, root crops are grown in the field in so-called “plots”. A plot represents a parcel of land of pre-measured size, and permits the cultivation of several crops, their number providing a statistical indication regarding the nature and distribution of crop yield. In the production of sugar beets one finds in general about 90 beets per plot. The plots is rated for productive capacity for sugar beets, and, after uprooting, the sugar beet is analyzed for content. Such an analysis takes place by means of conventional series techniques, which provide high accuracy. The goal however is to keep the cost of analysis to a minimum.

Structure and composition of the sample used for analysis are crucial for the accuracy of determining of the content. It should be in particular be taken into consideration that, due to genetic, crop cultivation, and above-all environment-contingent influences on growth, significant differences occur from plant to plant in the concentrations of quality-determining ingredients. Furthermore, a non-uniform distribution of concentration of the relevant constituents also is found within individual root crops such as beets and in the bodies or potato tubers. This heterogeneity of the object of analysis has led to high sampling requirements, which was solved for turnips and potatoes so far by the generation of so-called mash samples. Although the procedures have been continuously improved over time with respect to analysis of, for example, beet pulp (DL 26 11 636 B1) and potato mash (Ziolko and Jehle (2002), GIT Laboratory Journal 2000, 268-273) these mash-samples, since they represent only a sampling of the total population of the crops of a plot, are only limitedly representative. As a result of such non-representative sampling, significant distortions may occur in the measurement of ingredients.

Automated laboratories are known, in which ingredients are determined in a serial manner following extraction of pulp samples with aluminum sulfate or lead acetate. In addition, near-infrared spectroscopy (NIRS) has proven to be useful in the analysis of ingredients from crops tested in analytical laboratories, which is carried out for mashed raw potato samples, samples of potato pulp, beet pulp samples, technical juices and special byproducts of sugar production from beets (Haase (2006), Starch-Stärka Vol 58 (6), 268-273; Heppner et al. (2000), Sugar Industry, 125 No. 5, 325-330; Fernandez et al. (2008), Journal of Near Infrared Spectroscopy 16, 105-110). This spectroscopic method makes it possible to determine several analytes simultaneously in a sample, provides a quick availability of results and avoids the use of reagents; it thus reduces the cost and time of an analysis.

The use of NIRS as an analytical measurement method for the determination of ingredients in root crops has so far been restricted to the laboratory environment and therefore has the disadvantage that in addition to the actual analysis, a number of other preparatory sample treatment steps are needed, including activities such as fall harvesting, cleaning, collecting, storage, packing, labeling, freezing and sending of samples to the investigating laboratory. This increases the cost and the time required for analysis as a whole.

For cereals, maize and grass, NIR spectroscopy has already been used for real-time analysis of substances in conjunction with harvesting machines (WO 99/58959 Al). Here, a near infrared (NIR) probe composed of directed light source and sensor is oriented towards the flow of harvested materials, which consists of cereal grains, or even harvested chopped corn or grass chaff.

In practice, however, it has been found that a lack of controllability over the chopped materials with this method a separation can already begin to take place prior to the analysis, as a result of which distortions of the analytical results have occurred. Besides this, the known harvesting machines are not suited for analysis of root crops of individual parcels.

The present invention is therefore concerned with the task of specifying a method and apparatus for the determination of ingredients in a plot of root crops, with a high analysis accuracy and a short duration of analysis, and where the resulting data are representative for all root crops of one plot.

According to the invention the task is solved by the following sequence of steps:
a) finely dividing the root crops of a plot into substantially equal sized fine pieces,
b) generating a stream of fine pieces of root crop, and transporting the fine pieces of root crop with the aid of a transport device,
c) homogenizing or making uniform the stream of fine pieces of root crop,
d) irradiating the stream of fine pieces of root crop with light of the near infrared range,
e) recording the reflected radiation,
f) converting radiation into a spectral signal,
g) processing of the spectral signal for determination of the components.

With this invention it became possible for the first time to ensure an optimized representativeness of the sample from root crops, wherein the biological variability of the crop and the heterogeneous distribution of ingredient in the plant body is taken into account. Another advantage of the invention lies in the fact that the root crops are reduced to fine pieces just before the step of homogenizing or evening out of the stream, resulting in an additional increase in precision of the analytical result, since long transport route between the site of harvesting and the site of analysis is avoided. In this way a particularly good homogeneity of the test sample can be achieved.

Overall, the present invention therefore provides a method and a device enabling a quick processing of several parcels in succession, and thus enable a high analysis throughput, while at the same time also being easy to use.

According to a preferred embodiment of the invention is therefore possible to determine the contents present in the collective root crops of a plot. It is therefore not necessary to collect samples from individuals, which would significantly extend the time required for analysis.

To analyze the ingredients of the root crops, the root crops of a plot are harvested, the leaves are removed if necessary, and the harvested crop is cleaned. Then the root crops are introduced into a grinding device, where the centers of the root crops are reduced or ground into pieces of about 10 to 25 mm.

The transport of the reduced root crops can be done using a transport device, preferably in the form of a conveyor belt.

With the help of the transport device, the crushed root crops are transported to an analysis unit, for measuring the contents. Before the measurement, however, the stream of crushed root crops is evened or homogenized. This has the advantage that creates a stability against vibration, and that a leveled surface results, which provides a good surface for the subsequent measurement of the contents. A comparative homogenization and smoothing of the sample flow is possible through a device with a roller. Therein, the roller is arranged with its roller axis at a fixed and constant distance above the transporting device. The sample stream of ground-up root crop is compressed using the roller to a certain thickness and the surface becomes a smooth surface.

Preferably, the roller is mounted to rotate around it's longitudinal axis. It can be driven to rotate by the sample stream or by a motor. In the event of a motor, the roller can be driven with or counter to the stream of crushed root crops.

Subsequently, the sample stream is directed to a measuring device that has a near-infrared sensor head with a light source and a sensor. The surface of the sample stream is irradiated with the light in the near infrared range. Preferably, this irradiation is distributed over the entire surface of the sample flow, in order to homogenize the results. The radiation reflected by the sample stream is a function of the components to be measured. Subsequently, the sensor receives the reflected radiation, and performs a transformation of the received radiation into spectral signals either continuously or stepwise at preset intervals. These absorption spectra are generated in the wavelength range from 850 nm to 1650 nm and digitally filtered.

According to another embodiment of the invention, the light source and the sensor are pivotably arranged relative to the flow of the crushed root crops.

The device for carrying out the inventive method can be used almost anywhere. It therefore offers the advantage of being able to perform analysis on the spot, i.e., in close proximity to a parcel. For this purpose it is good practice to mount the device on a mobile platform, for example, on a vehicle. In connection with, for example, a sugar beet harvester or potato harvester, in this manner the necessary analysis can be carried out immediately following harvest, without requiring intermediate storage of the harvested root crops or the need for laborious transport.

The inventive method is described below in greater detail using a preferred embodiment of a method for the determination of components in root crops.

In the FIGURE, a device for the determination of ingredients in root crops is shown schematically: Cleaned root crops of a parcel are collected in a funnel-shaped reservoir 13. From the reservoir the root crops move to a reducing device 14. In the reducing device 14, the root crops are reduced using a cutting apparatus 15 into essentially even-sized pieces. For this, the cutting apparatus 15 is provided with two motor-driven, counter rotating shafts, which are fitted with hooks. Of these two shafts only one shaft 16 is shown in the FIGURE. The hooks of the shafts grab the fed root crops and lead them to a not-illustrated cutting element located between the shafts via which the root crops are reduced in size.

The root crop pieces produced by the reducing device 14 fall onto a conveyor belt 5 and accumulate there. The speed of the conveyor belt 5 is adjustable and is adapted to the speed at which the root crops are reduced, however the result of the accumulation of pieces from the reducing device 14 on the conveyor belt 5 does not result in a smooth surface. On the conveyor belt 5, the accumulated root crop pieces therefore go into a device 3, which provides a comparative even distribution of the sample flow. The device 3 has a roller 6 in the form of an elongate shaft, which is arranged a constant and fixed distance above the conveyor belt 5 along the roll axis 7. Using this roller 6, the sample stream of chopped root crops are compressed to a certain thickness whereby a smooth surface results. The distance between the roller 6 and the conveyor belt 5 is adjustable. It is preferably between 100 mm and 150 mm.

An electric motor, not shown in the FIGURE, drives the rollers 6 and rotates them in the running direction of the conveyor belt 5.

As the chopped root crops contact the roller 6, they are spread on the conveyor belt 5 and are subject to a compressive force as a function of the distance between the roller 6 and the conveyor belt 5. The so-compressed sample of root crop thus has imparted to it a smooth surface and a constant height.

Scrapers 8, 19 are provided on the roller 6 and the conveyor belt 5 and continuously clean the roller surface and belt during operation, thus avoiding the cross-mixing of two root crop samples of consecutive processed plots. Moreover, a clumping or accumulation of root crop sample on the conveyor belt 5 and the roller 6 can be ruled out, which would otherwise severely disturb the comparative homogenization of the sample flow.

Directly downstream of the roller 6 is a sensor head 9 with a light source 10 and a sensor 11 for detecting the radiation reflected from the smooth surface of the stream of root crop sample in the wavelength range from 850 nm to 1650 nm. The sensor head 9 is elevated at a fixed distance of 200 to 250 mm to the surface of the smooth sample flow and can be pivoted as desired relative to the sample stream. In this way, it is possible to sense and record the entire width of the sample stream.

The sensor 11 continuously records reflected radiation and transmits it via optical fiber 17 to a spectrometer 18, which converts the spectrally resolved radiation wavelengths into digitized, the spectral signals at regular intervals of 40 ms. Thus, during the flow-by of the stream of root crop samples, several hundred such spectra are produced, which are filtered and averaged by a processor 12. By comparison with suitable calibration data, the identities and concentrations of quality-ingredients such as sugar or starch are determined with high precision and are output.

Following the analysis, the reduced pieces of root crop can be distributed as an organic deposit in the plot, or fed as a raw material to a suitable reactor for high-quality biogas production.

### LIST OF REFERENCE NUMBERS

1 device
2 transporter
3 roller device
4 measuring system
5 conveyor belt
6 roller
7 roller axis
8 wiper blade
9 sensor head
10 light source
11 sensor
12 processor
13 reservoir
14 reducing device
15 cutting apparatus
16 shaft
17 fiber optic cable
18 spectrometer
19 wiper blade

1. A process for the determination of components in root crops comprising the following sequence of steps:
a) finely dividing the root crops of a plot into substantially equal sized fine pieces,
b) generating a stream of fine pieces of root crop, and transporting the fine pieces of root crop with the aid of a transport device,
c) homogenizing or evenly distributing the fine pieces of root crop in the stream,
d) irradiating the stream of fine pieces of root crop with light of the near infrared range,
e) recording the reflected radiation,
f) converting radiation into a spectral signal,
g) processing of the spectral signal for determination of the components.

2. The process of claim 1, wherein all the root crops of a plot are used in the determination of the components.

3. The process of claim 1, wherein the transport device of step b) is a conveyor belt.

4. The process of claim 1, wherein a stream of root crop pieces is spread out and smoothed.

5. The process of claim 4, wherein the entire surface of the stream of root crop pieces is irradiated with light of the near infrared range and the reflected radiation is detected.

6. The process of claim 1, wherein the conversion of the received radiation into spectral signals occurs continuously or at predetermined intervals.

7. The process of claim 1, wherein said absorption spectra are produced in the wavelength range from 850 nm to 1650 nm, and digitally filtered.

8. A device (1) for performing the process according to claim 1, the device (1) including a device (14) for reducing the root crop to fine pieces, a transport device (2), a device (3) to equalize a stream of reduced root crops and a measuring device (4) for identification and quantification of ingredients.

9. The device (1) according to claim 8, wherein the transport device is a conveyor belt (5).

10. The device (1) according to claim 8, wherein the device (3) to equalize the stream of reduced root crop is a roller (6).

11. The device (1) according to claim 10, wherein the roller (6) along the roller axis (7) is arranged at a fixed and constant distance above the transport device (2).

12. The device (1) according to claim 10, wherein the roller (6) is mounted rotatably around its longitudinal axis (7).

13. The device (1) according to claim 12, wherein the roller (6) is motorized for rotation with or against the stream of reduced root crops.

14. The device (1) according to claim 8, wherein the measuring device has a near-infrared sensor head (9) with a light source (10) and a sensor (11), wherein the light source (10) and the sensor (11) are pivotably arranged relative to the flow of the crushed root crop.

15. The device (1) according to claim 8, wherein the device (1) is connected to harvester for root vegetables.
